(19) United States
(12) Patent Application Publication
(10) Pub. No.: US 2026/0085028 A1
(43) Pub. Date: Mar. 26, 2026

(54) P-XYLENE CYCLIC DIMER DERIVATIVE FOR PREPARING PARYLENE FILM, AND PREPARATION METHOD AND USE THEREOF

(71) Applicants: SOOCHOW UNIVERSITY, Suzhou (CN); SUZHOU KARY NANO TECH CO., LTD., Suzhou (CN)

(72) Inventors: Jianping LANG, Suzhou (CN); Jie Zhou, Suzhou (CN); Rongjuan DAI, Suzhou (CN); Chunyan NI, Suzhou (CN); Lei CAO, Suzhou (CN); Jianqiang SHEN, Suzhou (CN); Xinghua WU, Suzhou (CN); Shenhao XU, Suzhou (CN); Xiang LI, Suzhou (CN)

(21) Appl. No.: 19/400,852

(22) Filed: Nov. 25, 2025

Related U.S. Application Data
(63) Continuation of application No. PCT/CN2025/082370, filed on Mar. 13, 2025.

(30) Foreign Application Priority Data
Sep. 23, 2024 (CN) 202411321347.6

(51) Int. Cl. C07C 17/269; B05D 1/00

(52) U.S. Cl. CPC C07C 17/269; B05D 1/60

(57) ABSTRACT

The invention provides a p-xylene cyclic dimer derivative for preparing a parylene film, and a preparation method and use thereof. The method includes: reacting 4-methyl-2-(trifluoromethyl)benzoic acid with a reducing agent in the presence of a solvent, to obtain an intermediate 1; reacting the intermediate 1 with a chlorinating agent in the presence of a solvent, to obtain an intermediate 2; reacting the intermediate 2 with an organic amine in the presence of a solvent, to obtain an intermediate 3; and reacting the intermediate 3 with a base in the presence of a polymerization inhibitor and a solvent, to obtain 4,16-bis(trifluoromethyl)-[2,2]-p-xylene cyclic dimer. A parylene film prepared with the compound as a monomer shows a high hydrophobicity that is superior to the optimal waterproof parylene AF4 commercially available at present. The preparation cost is low, so the present invention has good application prospects in protective materials.

This application is a Continuation of PCT/CN2025/082370, filed on Mar. 13, 2025, which claims priority to Chinese Patent Application No. 202411321347.6, filed on Sep. 23, 2024, which is incorporated by reference for all purposes as if fully set forth herein.

### FIELD OF THE INVENTION

The present invention relates to the technical field of organic synthesis, and specifically to a p-xylene cyclic dimer derivative for preparing a parylene film, and a preparation method and use thereof.

### DESCRIPTION OF THE RELATED ART

The poly-p-xylene family prepared with a p-xylene cyclic dimer and derivatives thereof is a class of protective materials with excellent performances. This class of materials play an important role in the three-proof materials because of their no need of solvent and initiator, high purity, good flexibility, being smooth and pinhole free, and good conformality. Different p-xylene derivatives can bring new characteristics on the basis of the original excellent properties, which makes the poly-p-xylene materials have a very broad scope of applications (see: C. P. Tan, H. G. Craighead, Materials, 2010, 3, 1803-1832) and have outstanding performances in microelectromechanical systems, electronic components, biomedicine, protection and other application scenarios.

This class of p-xylene cyclic dimers and their derivatives can be conformally deposited on any material and any complex geometry without pinholes by chemical vapor deposition (CVD) to form a parylene film, to provide an excellent barrier for protection against water diffusion and corrosion. Therefore, parylene films have a broad scope of applications in any technical areas that require definite dielectric, hydrophobic and chemical barriers (see: C. Desai, N. Laube, J. Coat. Technol. Res., 2019, 16 (1), 103-111). For example, in the biomedical field where the parylene films receive great attention, surface wettability is one of the key characteristics of parylene films, which affects the biological reactions on the micro-device biological interface, such as cell adhesion, protein adsorption and blood coagulation (see: X. P. Bi, B. P. Crum, W. Li, J. Microelectromech. Systems, 2014, 23(3), 628-635). At present, hydrophobic parylene includes parylene C, parylene D, and parylene AF4 etc., which have a contact angle with water of 87°, 97°, 100° respectively when untreated (see: B. J. Raos, M. C. Simpson, C. S. Doyle, E. S. Graham, C. P. Unsworth, PLoS One, 2019, 14(6), e0218850). The water contact angle of this class of parylene films is usually not higher than 100, and the monomer of parylene AF4 with relatively better hydrophobic performance is expensive, and costs as high as $20,000 per kilogram (see: G. Lee, H. J. Lee, J. Lee, K. J. Lee, Thin Solid Films, 2024,797,140333, which greatly limits the practical use of the parylene films in various areas.

Based on this, a monomer for preparing a parylene film with low production cost and good hydrophobicity is urgently needed to promote the use of the parylene films in various areas.

### SUMMARY OF THE INVENTION

The present invention provides a p-xylene cyclic dimer derivative for preparing a parylene film and a preparation method and use thereof. The parylene film prepared with 4,16-bis(trifluoromethyl)-[2,2]-p-xylene cyclic dimer as a monomer shows a high hydrophobicity that is superior to parylene AF4. The compound is prepared by reduction, chlorination, salt forming, and cyclization with 4-methyl-2-(trifluoromethyl)benzoic acid as a starting material. The operation is simple and the preparation cost is low, so the present invention has good application prospects in protective materials.

To solve the above technical problems, the following technical solutions are adopted in the present invention.

In a first aspect, the present invention provides a p-xylene cyclic dimer derivative for preparing a parylene film. The p-xylene cyclic dimer derivative has the following structure:

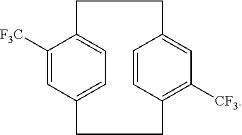 [SMILES: FC(F)(F)c1cc2ccc1CCc1ccc(c(C(F)(F)F)c1)CC2]

In a second aspect, the present invention provides a method for preparing a p-xylene cyclic dimer derivative for preparing a parylene film according to the first aspect. The preparation method includes the following steps:
S1: reacting 4-methyl-2-(trifluoromethyl)benzoic acid with a reducing agent in the presence of a first solvent, to obtain an intermediate 1;
S2: reacting the intermediate 1 with a chlorinating agent in the presence of a second solvent, to obtain an intermediate 2;
S3: reacting the intermediate 2 with an organic amine in the presence of a third solvent, to obtain an intermediate 3; and
S4: reacting the intermediate 3 with a base in the presence of a polymerization inhibitor and a fourth solvent, to obtain the p-xylene cyclic dimer derivative.

The intermediates 1-3 have the structures respectively as shown below:

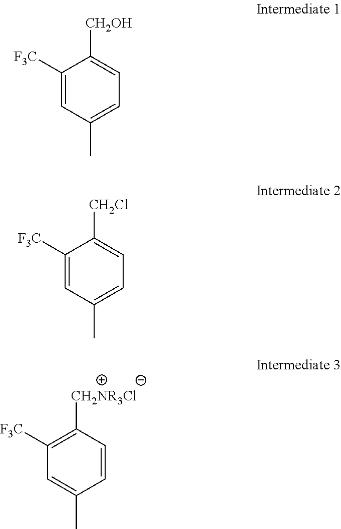

in which, R is methyl, ethyl or propyl.

Further, in S1, the reducing agent is preferably lithium aluminum hydride and/or sodium borohydride; further preferably, the molar feed ratio of 4-methyl-2-(trifluoromethyl)benzoic acid to the reducing agent is (0.5-3):1, for example, 0.5:1, 1:1, 1.5:1, 2:1, 2.5:1, or 3:1, including, but not limited to, the molar ratios listed above. Further, in S1, the first solvent is selected from the group consisting of diethyl ether, tetrahydrofuran, dichloromethane, toluene and any combination thereof. In some preferred embodiments, the first solvent is diethyl ether; and further preferably, the weight ratio of 4-methyl-2-(trifluoromethyl)benzoic acid to the first solvent is (0.01-0.2):1.

Further, in S1, the reaction temperature is preferably 30-60° C., and the reaction time is preferably 6-24 h.

Further, in S1, 4-methyl-2-(trifluoromethyl)benzoic acid is dissolved in the first solvent, and then the reducing agent is added at 0-30° C., heated to 30-60° C., and reacted for 6-24 h.

Further, in S1, the preparation method further includes the steps of: cooling to 0-5° C. after the reaction, adding water and/or a sodium hydroxide aqueous solution to quench the reaction, filtering to remove the precipitate, and drying the organic phase to obtain the intermediate 1.

Further, in S2, the chlorinating agent is preferably selected from the group consisting of phosphorus trichloride, phosphorus, thionyl chloride, concentrated hydrochloric acid, Lucas reagent and any combination thereof; and further preferably, the molar feed ratio of the intermediate 1 to the chlorinating agent is (0.5-3):1, for example, 0.5:1, 1:1, 1.5:1, 2:1, 2.5:1, or 3:1, including, but not limited to, the molar ratios listed above.

Further, in S2, the second solvent is selected from the group consisting of dichloromethane, diethyl ether, tetrahydrofuran, toluene and any combination thereof.

In some embodiments, the second solvent is dichloromethane; and further preferably, the weight ratio of the intermediate 1 to the second solvent is (0.01-0.4):1.

Further, in S2, the reaction temperature is preferably 30-60° C., and the reaction time is preferably 1-3 h.

Further, in S2, the intermediate 1 is dissolved in the second solvent, and then the chlorinating agent is added at 0-30° C., heated to 30-60° C., and reacted for 1-3 h.

Further, in S2, the preparation method further includes the steps of: cooling to 0-20° C. after the reaction, adding a saturated sodium bicarbonate solution to quench the reaction, extracting, and evaporating to obtain the intermediate 2.

Further, in S3, the organic amine is preferably trimethyl amine, triethyl amine, or tripropyl amine; and further preferably, the molar feed ratio of the intermediate 2 to the organic amine is (0.5-2.5):1, for example, 0.5:1, 1:1, 1.5:1, 2:1, or 2.5:1, including, but not limited to, the molar ratios listed above.

Further, in S3, the third solvent is toluene; and further preferably, the weight ratio of the intermediate 2 to the third solvent is (0.2-0.5):1.

Further, in S3, the reaction temperature is preferably 40-80° C., and the reaction time is preferably 3-8 h.

Further, in S3, the intermediate 2 is dissolved in the third solvent, and then the organic amine is added, then heated to 40-80° C., and reacted for 3-8 h.

Further, in S3, the preparation method further includes the steps of: standing after the reaction, collecting the aqueous layer, washing, and drying, to obtain the intermediate 3.

Further, in S4, the base is preferably selected from the group consisting of sodium hydroxide, potassium hydroxide, sodium ethoxide, potassium ethoxide and any combination thereof, and the polymerization inhibitor is preferably selected from the group consisting of hydroquinone monobutyl ether, tert-butyl catechol, p-tert-butyl phenol and any combination thereof; and further preferably, the molar feed ratio of the intermediate 3, the base, and the polymerization inhibitor is (20-50):(150-200):1.

Further, in S4, the fourth solvent is preferably selected from the group consisting of water, toluene, N, N-dimethylformamide, N,N-dimethylacetamide, sulfolane, benzene, xylene and any combination thereof.

Further, in S4, the reaction temperature is preferably 80-120° C., and the reaction time is preferably 2-24 h.

Further, in S4, the intermediate 3 is dissolved in water to obtain an aqueous solution of the intermediate 3, and then the aqueous solution of the intermediate 3 is added dropwise to a mixed solution of the base, the polymerization inhibitor and the fourth solvent at 80-100° C., and then heated to 100-120° C., and reacted for 2-24 h; and preferably, the weight ratio of the intermediate 3 to water is (0.1-0.8):1.

Further, in S4, the preparation method further includes the steps of: adding water and separating the organic phase after the reaction, washing the organic phase with water and/or an acid solution, adding activated carbon to the washed organic phase, heating to 40-100° C., stirring for 0.5-3 h, filtering, and crystallizing by cooling, to obtain the p-xylene cyclic dimer derivative. Preferably the solid crystallized by cooling is dissolved in n-hexane and recrystallized to obtain a purified product of the p-xylene cyclic dimer derivative.

The present invention further provides a parylene film, prepared by chemical vapor deposition of the p-xylene cyclic dimer derivative according to the first aspect as a monomer.

Compared with the prior art, the present invention has the following beneficial effects:

1. The present invention provides a p-xylene cyclic dimer derivative for preparing a parylene film, that is, 4,16-bis(trifluoromethyl)-[2,2]-p-xylene cyclic dimer. The parylene film prepared with the compound as a monomer has a water contact angle up to 105°, which is obviously advantageous over the commonly used hydrophobic parylene and advantageous over the currently commercially available optimal hydrophobic parylene AF4.

2. The present invention further provides a method for synthesizing 4,16-bis(trifluoromethyl)-[2,2]-p-xylene cyclic dimer by reduction, chlorination, salt forming, and cyclization with inexpensive 4-methyl-2-(trifluoromethyl)benzoic acid as a starting material. The synthesis method is simple in operation, the reaction conditions are mild, the product yield is high, the preparation cost is low, and the cost of laboratory production is only 20 yuan (the cost can be further reduced in mass production), so the method is suitable for use in mass production. Compared with the expensive hydrophobic parylene AF4 monomer, inexpensive 4,16-bis(trifluoromethyl)-[2,2]-p-xylene cyclic dimer can be prepared, which has a good aspect of application in protective materials.

### BRIEF DESCRIPTION OF THE DRAWINGS

FIG. 1 shows a reaction scheme for preparing 4,16-bis(trifluoromethyl)-[2,2]-p-xylene cyclic dimer;

FIG. 2 shows a ¹H NMR spectrum of 4-methyl-2-(trifluoromethyl)benzyl alcohol;

FIG. 3 shows a ¹³C NMR spectrum of 4-methyl-2-(trifluoromethyl)benzyl alcohol;

FIG. 4 shows a ¹H NMR spectrum of 4-methyl-2-(trifluoromethyl)benzyl chloride;

FIG. 5 shows a ¹³C NMR spectrum of 4-methyl-2-(trifluoromethyl)benzyl chloride;

FIG. 6 shows a ¹H NMR spectrum of 4-methyl-2-(trifluoromethyl)benzyltrimethyl-ammonium chloride;

FIG. 7 shows a ¹³C NMR spectrum of 4-methyl-2-(trifluoromethyl)benzyltrimethyl-ammonium chloride;

FIG. 8 shows a ¹H NMR spectrum of 4,16-bis(trifluoromethyl)-[2,2]-p-xylene cyclic dimer;

FIG. 9 shows a ¹³C NMR spectrum of 4,16-bis(trifluoromethyl)-[2,2]-p-xylene cyclic dimer;

FIG. 10 shows the test result for the contact angle of a parylene film prepared with 4,16-bis(trifluoromethyl)-[2,2]-p-xylene cyclic dimer as a monomer; and

FIG. 11 compares the contact angles with water of parylene films prepared with different monomers.

FIG. 12 shows the parylene film prepared by the supplementary example 1.

FIG. 13 shows the contact angle of the parylene film prepared by the supplementary example 1.

### DETAILED DESCRIPTION OF THE PREFERRED EMBODIMENTS

Unless otherwise defined, all technical and scientific terms used herein have the same meaning as commonly understood by persons skilled in the art. The terms used in the description of the present invention are for the purpose of describing particular embodiments only and are not intended to limit the present invention. The term “and/or” as used herein includes any and all combinations of one or more of the listed related items. As used herein, the term “including” or “comprising” may indicate that it is possible to include or comprise other components in addition to the components mentioned. As used herein, the term “including” or “comprising” can also be replaced by the closed “being” or “consisting of”.

The present invention will be further described below in connection with specific examples and drawings, so that those skilled in the art can better understand and implement the present invention; however, the present invention is not limited thereto.

### Example 1

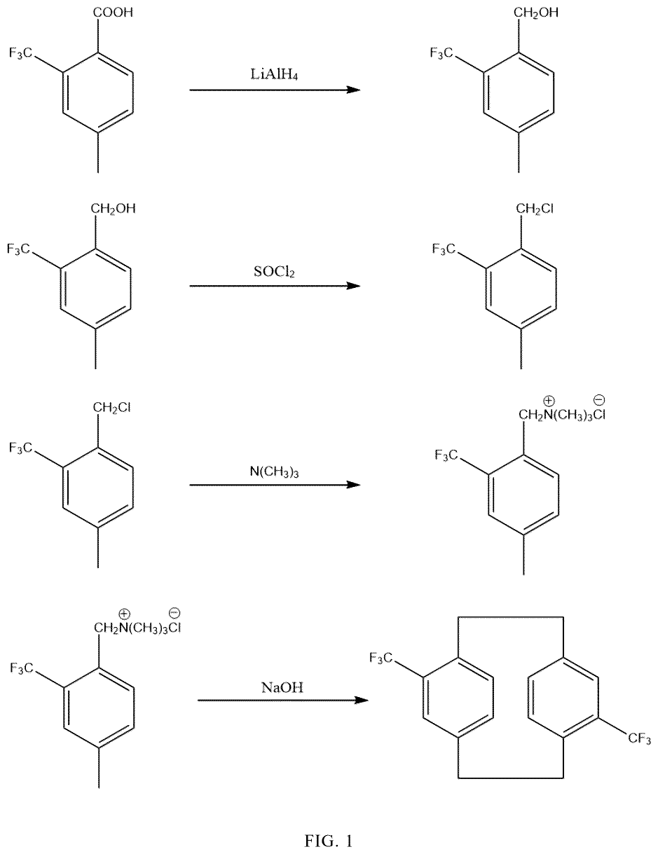

This example involves the preparation of 4,16-bis(trifluoromethyl)-[2,2]-p-xylene cyclic dimer. The reaction scheme is shown in FIG. 1 and specifically as follows:

### Preparation of Intermediate 1:

To a 500 mL three-neck flask, 15.31 g of 4-methyl-2-(trifluoromethyl)benzoic acid was added, and then 300 mL of diethyl ether was added as a solvent. The mixture was stirred for 30 min under a nitrogen atmosphere, and cooled to 0° C. Then, 45 mL of a lithium aluminum hydride solution (2.5 mol/L) in tetrahydrofuran was added, slowly heated to 40° C., heated to reflux, and continuously stirred for 24 h. After the reaction was completed, the reaction solution was cooled to 0° C., and the reaction was quenched by sequentially adding 4.3 mL of water, 4.3 mL of a 15% sodium hydroxide solution, and 12.9 mL of water. The precipitate was filtered off, and the organic phase was dried and removed of the solvent, to obtain 13.05 g of an intermediate 1 with a yield of 97.90%.

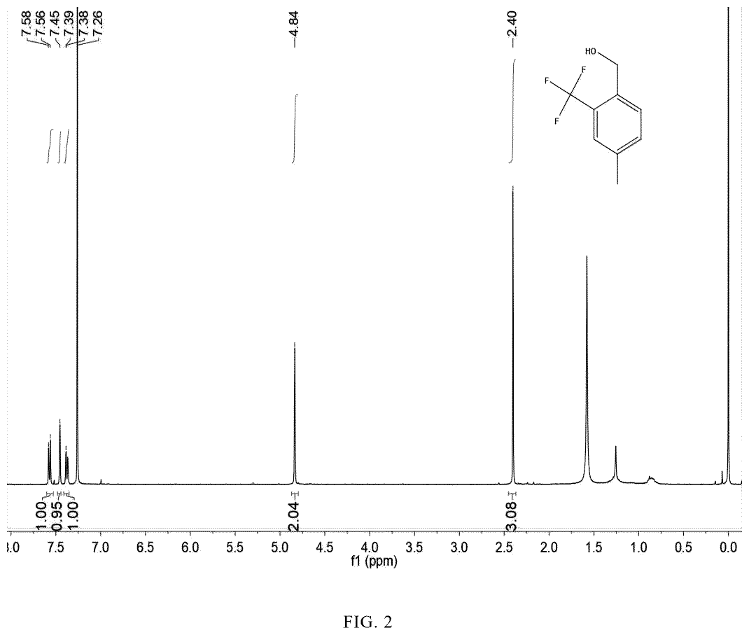

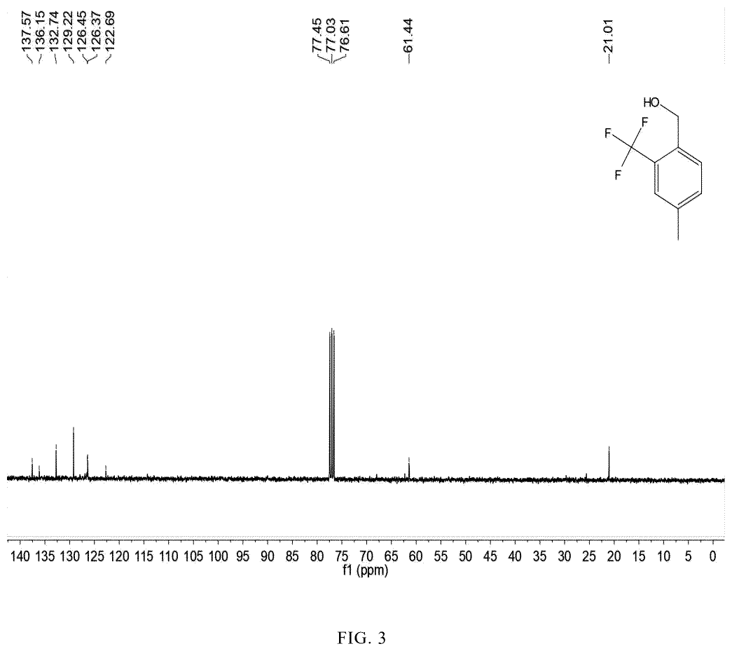

The prepared intermediate 1 was characterized by nuclear magnetic resonance spectroscopy, as shown in FIGS. 2 and 3. The specific results are shown below:

¹H NMR (400 MHz, CDCl₃): δ 7.57 (d, J=7.8 Hz, 1H) 7.45 (s, 1H), 7.38 (d, J=1.6 Hz, 1H), 4.84 (s, 2H), 2.40 (s, 3H).

¹³C NMR (75 MHz, CDCl₃): δ 137.57, 136.15, 132.74, 129.22, 126.45, 126.37, 122.69, 61.44, 21.01.

As can be seen from the characterization results, the intermediate 1 is 4-methyl-2-(trifluoromethyl)benzyl alcohol.

### Preparation of Intermediate 2:

12 g of the above prepared intermediate 1 was added to a 250 mL three-neck flask, and 120 mL of diethyl ether was added as a solvent. The mixture was stirred for 30 min under a nitrogen atmosphere, and cooled to 0° C. Then, 8 mL of thionyl chloride was added, slowly heated to 40° C., heated to reflux, and continuously stirred for 3 h. After cooling, the reaction solution was quenched with a saturated sodium bicarbonate solution, and allowed to stand. The organic phase was collected and evaporated to obtain 9.68 g of an intermediate 2 with a yield of 73.54%.

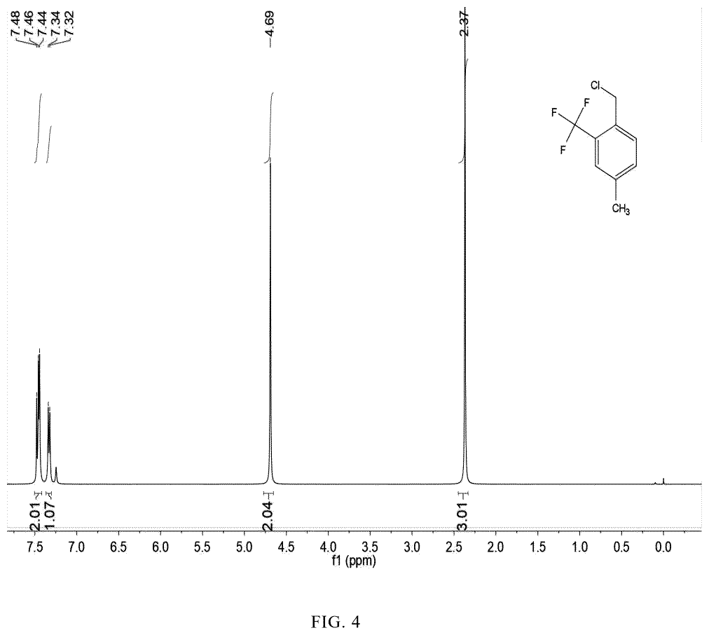

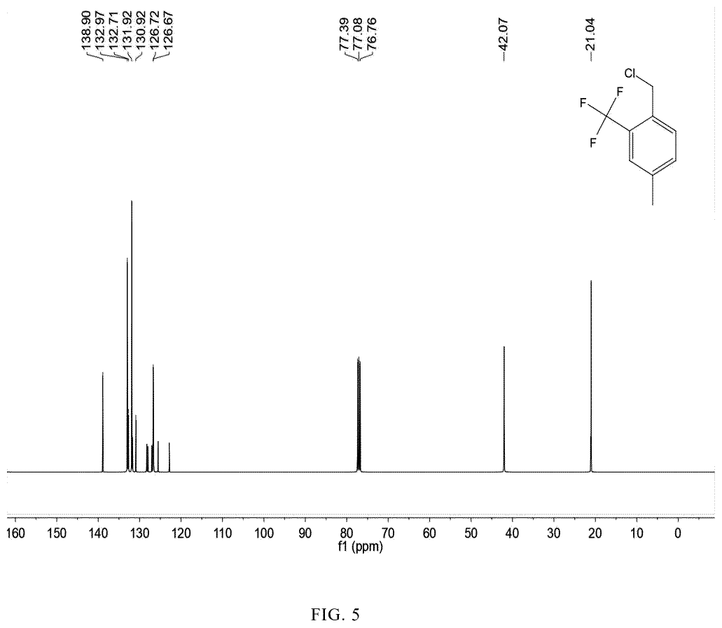

The prepared intermediate 2 was characterized by nuclear magnetic resonance spectroscopy, as shown in FIGS. 4 and 5. The specific results are shown below:

¹H NMR (400 MHz, CDCl₃): δ 7.47 (d, J=7.7 Hz, 1H), 7.44 (s, 1H), 7.33 (d, J=7.7 Hz, 1H), 4.69 (s, 2H), 2.37 (s, 3H).

¹³C NMR (100 MHz, CDCl₃): δ 138.90, 132.97, 132.71, 131.92, 130.92, 126.72, 126.67, 42.07, 21.04.

As can be known from the characterization results, the intermediate 2 is 4-methyl-2-(trifluoromethyl)benzyl chloride.

### Preparation of Intermediate 3:

To a 250 mL three-neck flask, 20 g of the intermediate 2, 65 mL of toluene, and 20 g of 30% trimethylamine in water were added, held at 35° C., and continuously stirred for 4 h. After standing, the aqueous layer was collected, and washed with t-butyl methyl ether. The aqueous layer was filtered through diatomaceous earth, and the solvent was removed to obtain 18.6 g of an intermediate 3 with a yield of 76.76%.

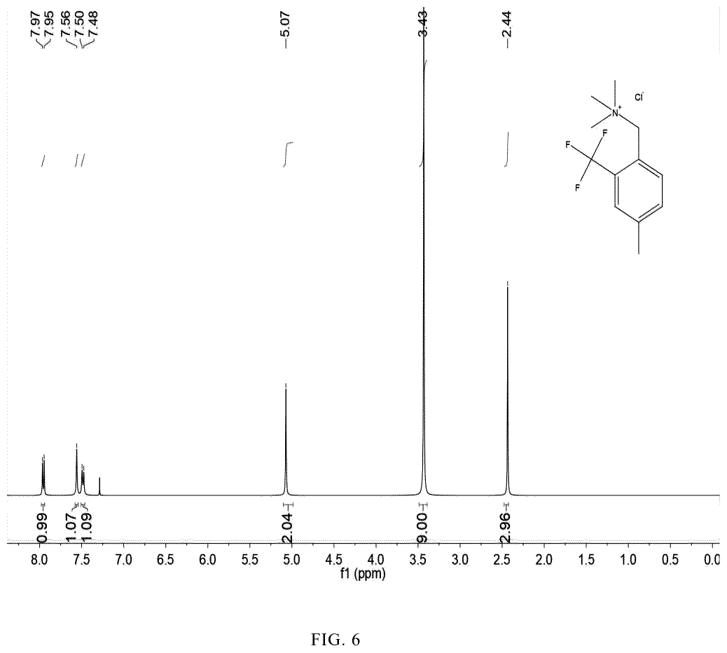

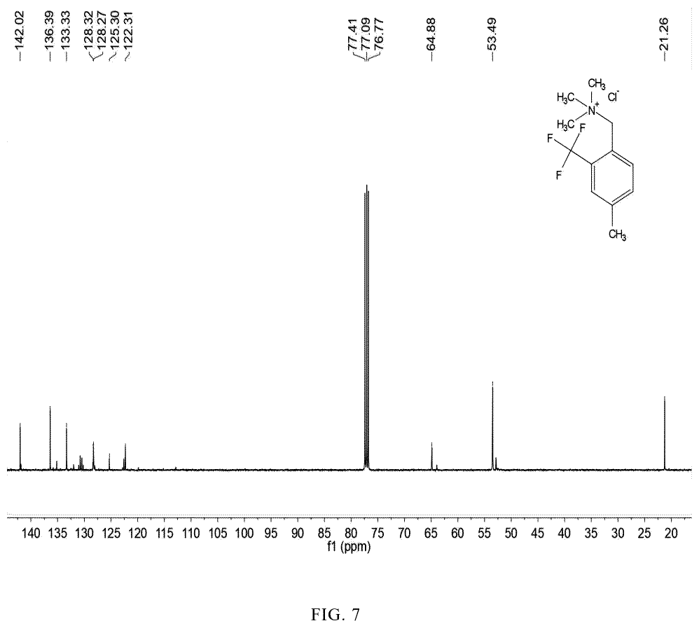

The prepared intermediate 3 was characterized by nuclear magnetic resonance spectroscopy, as shown in FIGS. 6 and 7. The specific results are shown below:

¹H NMR (400 MHz, CDCl₃): δ 7.96 (d, J=7.9 Hz, 1H), 7.56 (d, J=1.8 Hz, 1H), 7.49 (d, J=1.7 Hz, 1H), 5.07 (s, 2H), 3.43 (s, 9H), 2.44 (s, 3H).

¹³C NMR (100 MHz, CDCl₃): δ 142.02, 136.39, 133.33, 128.32, 128.27, 125.30, 122.31, 64.88, 53.49, 21.26.

As can be seen from the characterization results, the intermediate 3 is 4-methyl-2-(trifluoromethyl)benzyltrimethylammonium chloride.

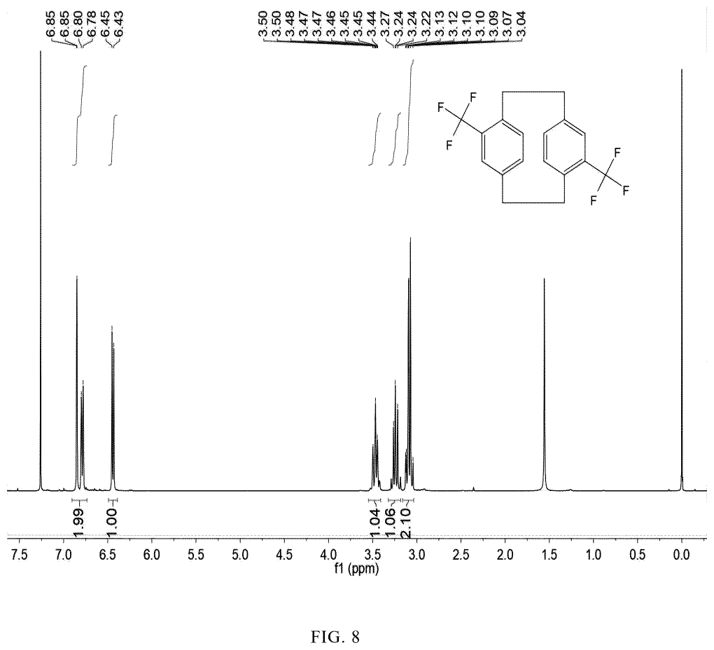

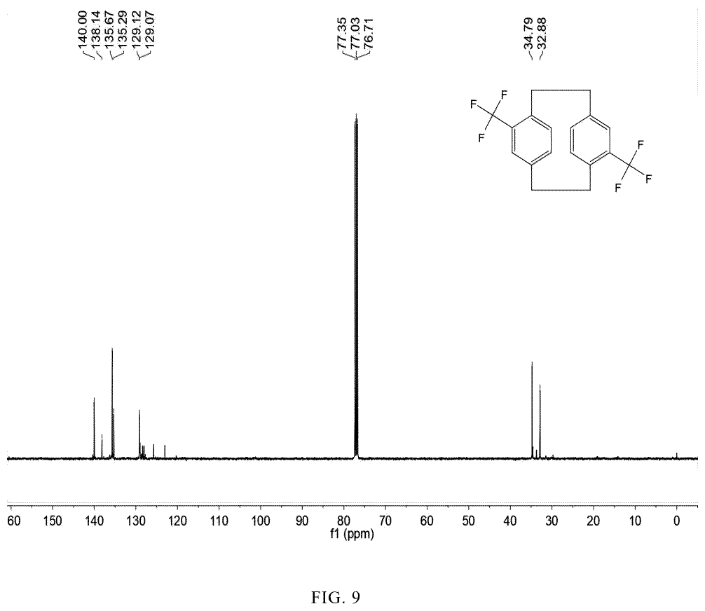

Preparation of 4,16-bis(trifluoromethyl)-[2,2]-p-xylene cyclic dimer: 20 g of the intermediate 3 was dissolved in 30 g of water, to obtain a quaternary ammonium salt solution for later use. To a three-neck flask, 20 g of solid sodium hydroxide and 50 g of water were added and prepared into a solution. Then, 100 mL of toluene and 0.8 g of p-tert-butyl phenol were added and heated to 95° C. Then, the quaternary ammonium salt solution was slowly added dropwise over about 2 h. The mixture was slowly heated to 110° C. and held for 8 h, during which about 20 mL of water was separated. After the reaction, the reaction solution was cooled to room temperature, added with 20 mL of water, and allowed to stand. A basic solution was separated. The toluene layer was washed respectively with 20 mL of water, 20 mL of dilute sulfuric acid, and 20 mL of iced water. The treated toluene layer was added with 10 g of activated carbon, heated to 80° C., stirred for 0.5 h, filtered while hot, and crystallized by cooling. The product was re-crystallized in 220 g of n-hexane, and dried to obtain 12.4 g of the final product with a yield of 36.1% and a purity of 99.5%. The prepared final product was characterized by nuclear magnetic resonance spectroscopy, as shown in FIGS. 8 and 9. The specific results are shown below:

¹H NMR (400 MHz, CDCl₃): δ 6.85 (d, J=1.9 Hz, 1H), 6.79 (d, J=7.9 Hz, 1H), 6.44 (d, J=7.9 Hz, 1H), 3.41-3.52 (m, 1H), 3.24 (dd, J=10.4, 8.7 Hz, 1H) 3.04-3.15 (m, 2H).

¹³C NMR (100 MHz, CDCl₃): δ 140.00, 138.14, 135.67, 135.29, 129.12, 129.07, 34.79, 32.88.

As can be seen from the characterization results, the final product is 4,16-bis(trifluoromethyl)-[2,2]-p-xylene cyclic dimer.

### Comparative Example 1

4,16-dibromo-[2,2]-p-xylene cyclic dimer was dissolved in N, N-dimethylacetamide, and methyl 2,2-difluoro-2-(fluorosulfonyl)acetate was added and reacted at 80° C. for 8 h in the presence of cuprous iodide as a catalyst. After filtration and washing, a crude product was obtained. About 30% of the product and about 40% of a mono-substituted by-product were produced, as shown by mass spectrometry. Column chromatography was used for separation. Because the polarities of the raw material, the product and the by-product were extremely small and very close, the product could not be separated and purified.

### Comparative Example 2

4,16-dibromo-[2,2]-p-xylene cyclic dimer was dissolved in methylpyrrolidone, and sodium trifluoroacetate was added and reacted at 140° C. for 72 h in the presence of cuprous iodide as a catalyst. After filtration and washing, a crude product was obtained. Only 10% of the product and about 20% of a mono-substituted by-product were produced, as shown by mass spectrometry. A large amount of unreacted raw material was separated by column chromatography. Because the polarities of the raw material, the product and the by-product were extremely small and very close, the product could not be separated and purified.

### Application and Performance Characterization

4,16-bis(trifluoromethyl)-[2,2]-p-xylene cyclic dimer (referred to as parylene DCF3) prepared in Example 1 and commercially available parylene monomers (parylene C monomer, parylene D monomer, and parylene AF4 monomer) were respectively prepared into a corresponding parylene film by a method having the following specific operations:

The parylene monomer was fed to an evaporation chamber, evaporated in the evaporation chamber at a vacuum level of 4 Pa (the temperature of the evaporation chamber was 180° C.), then cracked in a pyrolyzed furnace (the temperature of the cracking furnace was 680° C.), and finally deposited on a glass sheet in a coating chamber to form a transparent film. The formed film was peeled off from the glass sheet to obtain the parylene film.

Structure of the parylene DCF3 monomer:

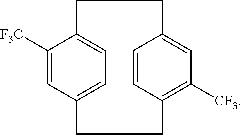 [SMILES: FC(F)(F)c1cc2ccc1CCc1ccc(c(C(F)(F)F)c1)CC2]

Structure of the parylene DCF3 film prepared:

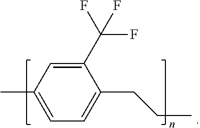

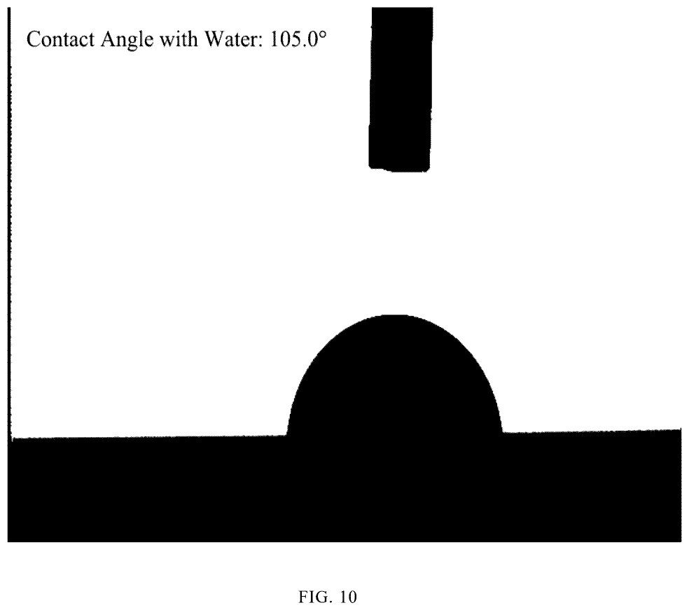

The test result of contact angle with water of the film is shown in FIG. 10, and the water contact angle can reach 105°.

Structure of the parylene C monomer:

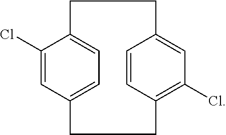 [SMILES: Clc1cc2ccc1CCc1ccc(c(Cl)c1)CC2]

Structure of the parylene C film prepared:

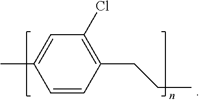

Structure of the parylene D monomer:

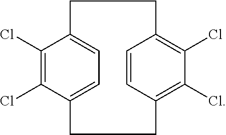 [SMILES: Clc1c2ccc(c1Cl)CCc1ccc(c(Cl)c1Cl)CC2]

Structure of the parylene D film prepared:

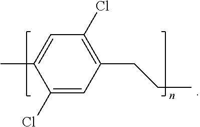

Structure of the parylene AF4 monomer:

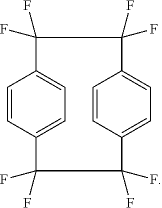 [SMILES: FC1(F)c2ccc(cc2)C(F)(F)C(F)(F)c2ccc(cc2)C1(F)F]

Structure of the parylene AF4 film prepared:

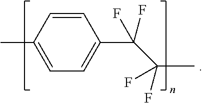

The test results of contact angle with water of the films prepared with various parylene monomers and the water contact angle of the parylene CCF3 film reported in a literature (G. Lee, H. J. Lee, J. Lee, K. J. Lee, Thin Solid Films. 2024, 797, 140333) are shown in Table 1 below. Structure of the parylene CCF3 monomer;

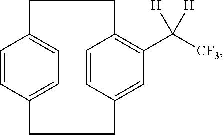 [SMILES: FC(F)(F)Cc1cc2ccc1CCc1ccc(cc1)CC2]

and structure of the film:

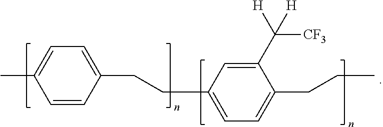

TABLE 1

| | Water contact angle |
|---|---|
| Parylene monomer | of parylene film (°) |
| Parylene DCF3 | 105 |
| Parylene C | 92.4 |
| Parylene D | 97.1 |
| Parylene AF4 | 100.3 |
| Parylene CCF3 | 92.6 |

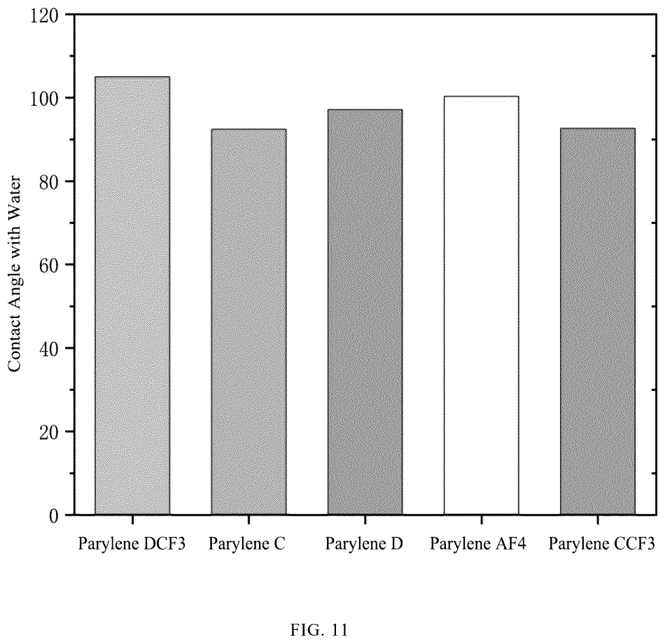

As demonstrated in Table 1 and FIG. 11, the parylene film prepared from the 4,16-bis(trifluoromethyl)-[2,2]-p-xylene cyclic dimer of the present invention exhibits a significantly higher water contact angle than films derived from conventional hydrophobic parylenes (e.g., Parylene C and D) and surpasses even the performance of the current benchmark, Parylene AF4. Furthermore, the monomer synthesis is substantially more cost-effective than that of Parylene AF4. The combination of superior hydrophobic performance and low production cost renders the present invention highly attractive and commercially competitive for a wide range of applications in hydrophobic protective coatings.

### Supplementary Example

S1. To 500 mL of dichloromethane, 0.5 mol of 3-(trifluoromethyl)-4-methylbenzyl bromide and 0.5 mol of N,N,N′,N′-tetramethyldiaminomethane were added sequentially. The mixture was reacted at 20° C. for 3 h under stirring. Subsequently, 100 mL of a dichloromethane solution containing 0.5 mol of 4-(bromomethyl)-1-methylbenzene was added dropwise, and the resulting solution was stirred for 3 h until the reaction was completed. After the reaction, the solvent was removed under reduced pressure to obtain a crude solid product. The solid was slurried with 200 mL of methyl tert-butyl ether to yield an intermediate product.

S2. To a reaction flask, an aqueous solution of 51% KOH (669 g, 6.09 mol of KOH), 1000 mL of xylene, 430 mL of dimethyl sulfoxide, and 0.004 mol of tetrabutylammonium bromide were added. 500 mL of an aqueous solution containing 0.4 mol of the intermediate product was added dropwise for 4 h under stirring at 90° C. After the addition, the reaction was carried out for 24 h when the internal temperature was maintained at 90° C. The reaction mixture was allowed to stand for phase separation, and the upper xylene phase was collected, washed with water, and decolorized with activated carbon. The filtrate was distilled and concentrated to 300 mL, cooled for crystallization to obtain white crystals. The crystals were vacuum-dried at 50° C. to obtain solid powder.

Preparation of Parylene Film: The parylene monomer prepared in the supplementary example 1 was evaporated under a vacuum degree of 4 Pa in an evaporation chamber at 180° C. The vapor was subsequently pyrolyzed in a pyrolysis furnace at 680° C. Finally, a transparent film was deposited onto a glass slide in a coating chamber. Then the film was peeled off from the glass slide, to obtain a parylene film with the following structure:

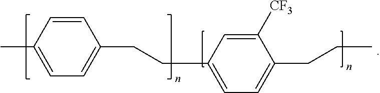

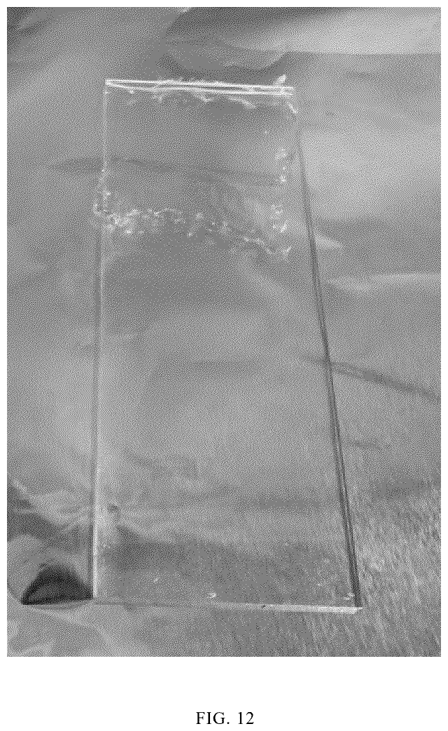

The hydrophobic contact angle of the prepared parylene film (FIG. 12) was tested.

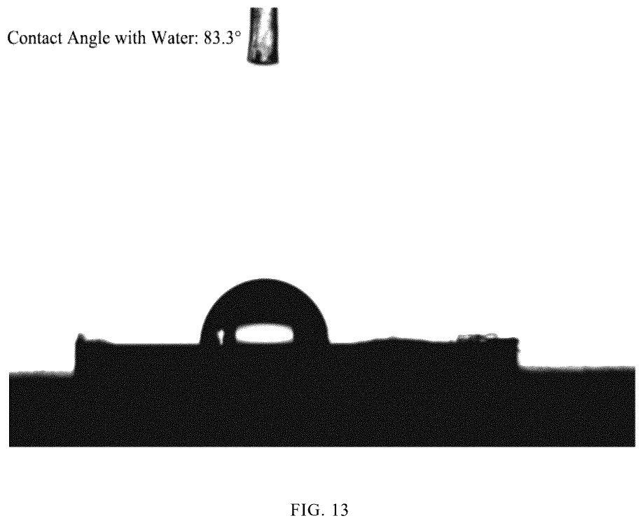

The test result is shown in FIG. 13. The film prepared by the product of supplementary example 1 exhibits a contact angle of only 83.3°, which is significantly lower than that of the film prepared from the parylene DCF3 (105°).

The above-described embodiments are merely preferred embodiments for the purpose of fully illustrating the present invention, and the scope of the present invention is not limited thereto. Equivalent substitutions or modifications can be made by those skilled in the art based on the present invention, which are within the scope of the present invention as defined by the claims. The scope of the present invention is defined by the appended claims.

What is claimed is:

1. A p-xylene cyclic dimer derivative for preparing a parylene film, the p-xylene cyclic dimer derivative having a structure of:

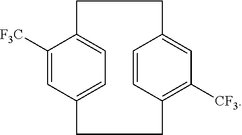 [SMILES: FC(F)(F)c1cc2ccc1CCc1ccc(c(C(F)(F)F)c1)CC2]

2. A method for preparing a p-xylene cyclic dimer derivative according to claim 1, comprising steps of:
S1: reacting 4-methyl-2-(trifluoromethyl)benzoic acid with a reducing agent in the presence of a first solvent, to obtain an intermediate 1;
S2: reacting the intermediate 1 with a chlorinating agent in the presence of a second solvent, to obtain an intermediate 2;
S3: reacting the intermediate 2 with an organic amine in the presence of a third solvent, to obtain an intermediate 3; and
S4: reacting the intermediate 3 with a base in the presence of a polymerization inhibitor and a fourth solvent, to obtain the p-xylene cyclic dimer derivative;
wherein the intermediates 1, 2 and 3 respectively have a structure as shown below: 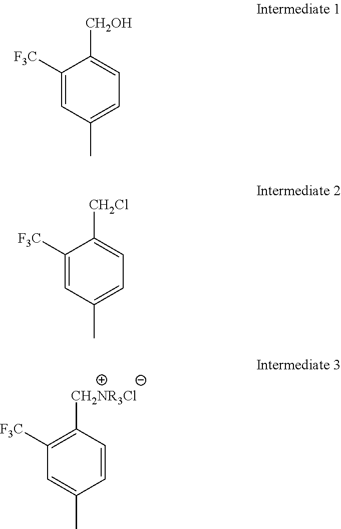
in which, R is methyl, ethyl or propyl.

3. The preparation method according to claim 2, wherein S1 at least comprises one of:
(1) the reducing agent is lithium aluminum hydride and/or sodium borohydride;
(2) a molar ratio of 4-methyl-2-(trifluoromethyl)benzoic acid to the reducing agent is (0.5-3):1;
(3) the first solvent is selected from the group consisting of diethyl ether, tetrahydrofuran, dichloromethane, toluene and any combination thereof; and
(4) the reaction temperature is 30-60° C., and the reaction time is 6-24 h.

4. The preparation method according to claim 2, wherein in S1,
4-methyl-2-(trifluoromethyl)benzoic acid is dissolved in the first solvent, and the reducing agent is added at 0-30° C., then the resulting solution is heated to 30-60° C. and reacted for 6-24 h; and
the preparation method further comprises steps of: cooling to 0-5° C. after the reaction, adding water and/or a sodium hydroxide aqueous solution to quench the reaction, filtering to remove the precipitate, and drying the organic phase to obtain the intermediate 1.

5. The preparation method according to claim 2, wherein S2 at least comprises one of:
(1) the chlorinating agent is selected from the group consisting of phosphorus trichloride, phosphorus pentachloride, thionyl chloride, concentrated hydrochloric acid, Lucas reagent and any combination thereof;
(2) a molar ratio of the intermediate 1 to the chlorinating agent is (0.5-3):1;
(3) the second solvent is selected from the group consisting of dichloromethane, diethyl ether, tetrahydrofuran, toluene and any combination thereof; and
(4) the reaction temperature is 30-60° C., and the reaction time is 1-3 h.

6. The preparation method according to claim 2, wherein in S2,
the intermediate 1 is dissolved in the second solvent, and then the chlorinating agent is added at 0-30° C., the resulting solution is heated to 30-60° C., and reacted for 1-3 h; and
the preparation method further comprises steps of: cooling to 0-20° C. after the reaction, adding a saturated sodium bicarbonate solution to quench the reaction, standing, collecting the organic layer, and evaporating to obtain the intermediate 2.

7. The preparation method according to claim 2, wherein S3 at least comprises one of:
(1) the organic amine is trimethyl amine, triethyl amine, or tripropyl amine;
(2) a molar ratio of the intermediate 2 to the organic amine is (0.5-2.5):1;
(3) the third solvent is toluene; and
(4) the reaction temperature is 40-80° C., and the reaction time is 3-8 h.

8. The preparation method according to claim 2, wherein in S3,
the intermediate 2 is dissolved in the third solvent, and then the organic amine is added, then the resulting solution is heated to 40-80° C. and reacted for 3-8 h; and
the preparation method further comprises steps of: standing after the reaction, collecting the organic layer, washing, and drying, to obtain the intermediate 3.

9. The preparation method according to claim 2, wherein S4 at least comprises one of:
(1) the base is selected from the group consisting of sodium hydroxide, potassium hydroxide, sodium ethoxide, potassium ethoxide and any combination thereof;
(2) the polymerization inhibitor is selected from hydroquinone monobutyl ether, tert-butyl catechol, p-tert-butyl phenol and any combination thereof;
(3) a molar ratio of the intermediate 3, the base, and the polymerization inhibitor is (20-50):(150-200):1;
(4) the fourth solvent is selected from water, toluene, N,N-dimethylformamide, N,N-dimethylacetamide, sulfolane, benzene, xylene and any combination thereof; and
(5) the reaction temperature is 80-120° C., and the reaction time is 2-24 h.

10. The preparation method according to claim 2, wherein in S4:
the intermediate 3 is dissolved in water to obtain an aqueous solution of the intermediate 3, and then the aqueous solution of the intermediate 3 is added dropwise to a mixed solution of the base, the polymerization inhibitor and the fourth solvent at 80-100° C., and then the resulting solution is heated to 100-120° C. and reacted for 2-24 h; and
the preparation method further comprises steps of: adding water and separating the organic phase after the reaction, washing the organic phase with water and/or an acid solution, adding activated carbon to the washed organic phase, heating to 40-100° C., stirring for 0.5-3 h, filtering, and crystallizing by cooling, to obtain the p-xylene cyclic dimer derivative.

11. A parylene film, prepared by chemical vapor deposition of the p-xylene cyclic dimer derivative according to claim 1 as a monomer.
